(19) United States
(12) Patent Application Publication
(10) Pub. No.: US 2020/0191928 A1
(43) Pub. Date: Jun. 18, 2020

(54) SYSTEMS AND METHODS FOR BEAMFORNING ULTRASOUND SIGNALS USING ELASTIC INTERPOLATION

(71) Applicant: KONINKLIJKE PHILIPS N.V., EINDHOVEN (NL)

(72) Inventors: David HOPE SIMPSON, BOTHELL, WA (US); Francois Guy Gerard Marie VIGNON, ANDOVER, MA (US); Andrew HANCOCK, SACRAMENTO, CA (US); Jun Seob SHIN, MEDFORD, MA (US)

(21) Appl. No.: 16/606,922

(22) PCT Filed: Apr. 16, 2018
(86) PCT No.: PCT/EP2018/059611

§ 371 Date: Oct. 21, 2019

Related U.S. Application Data
(60) Provisional application No. 62/488,924, filed on Apr. 24, 2017.

(51) Int. Cl. G01S 7/52; G10K 11/34; G01S 15/89

(52) U.S. Cl. CPC G01S 7/52025; A61B 8/12; G01S 15/8915; G10K 11/34

(57) ABSTRACT

A method in accordance with the present disclosure may include transmitting an ultrasound pulse toward a medium from a transducer array (305), detecting a plurality of echo signals (313a, 313c, 313e) responsive to the ultrasound pulse using one or more elements of the transducer array (305), generating an interpolated signal (313b, 313d) by interpolating a signal characteristic of at least two existing echo signals after temporally aligning the existing echo signals, and generating ultrasound image data based on one or more existing echo signals and the interpolated signal.

### TECHNICAL FIELD

This application relates to ultrasound imaging and more specifically to beamforming of ultrasound transmit and receive signals, for example using elastic interpolation.

### BACKGROUND

Ultrasound imaging is often performed by sequential insonification of a medium using focused beams. Each focused beam allows the reconstruction of a single image line. A 2D image is typically made of few tens or hundreds of lines and is created by the sequential reconstruction of each line in the image, the time for reconstructing each line depending on the image depth. Therefore, the time to build an image (e.g., frame rate) is dependent on the image depth and spatial resolution (e.g., number of image lines). Reconstructing an image line from signals received from elements of a transducer array is typically a computationally intensive process involving algorithms or specifically designed electronics to time the firing of the elements of the array and subsequent reception and processing of the received signals. In other examples, unfocused beams (e.g., plane waves) may be used to insonify a larger area with a single beam and reconstruct images at higher frame rates. A variety of synthetic aperture techniques that may involve single element transmits with diverging spherical waves may be used. Either way, with any existing techniques for insanitation and reconstruction of image data, improved methods and systems which reduce the number of elements in a transducer array or reduce the amount of times that transducer elements are fired for generating an image may be desired.

### SUMMARY

A method in accordance with the present disclosure may include transmitting one or more ultrasound pulses toward a medium from a transducer array, detecting a plurality of received echo signals responsive to the one or more ultrasound pulses using one or more elements of the transducer array, and generating an interpolated signal by interpolating a signal characteristic of at least two existing echo signals. The existing echo signals may include at least two echo signals selected from the plurality of received echo signals and previously interpolated echo signals, and the interpolating may be performed concurrently with or following temporal alignment of the at least two existing echo signals. The temporal alignment may be responsive to one or more features of the at least two existing echo signals, which may be a different from the signal characteristic being interpolated. The method may further include generating ultrasound image data based on one or more received echo signals and the interpolated signal.

In some examples of the method, the interpolating a signal characteristic of at least two existing echo signals may include calculating a respective envelope for each of the at least two echo existing echo signals, and estimating an envelope of the interpolated signal by interpolating between the envelopes of the at least two existing echo signals. In some examples, the temporal alignment may include estimating a temporal characteristic of the interpolated signal and aligning the interpolated signal relative to the at least two echo signals based on the temporal characteristic. In some examples, the temporally aligning the interpolated signal may include calculating a displacement vector for a respective envelope of each of the at least two echo signals, weighting the displacement vectors according to an interpolation factor, and averaging the weighted displacement vectors to generate the temporal characteristic of the interpolated signal. In some examples of the method, the calculating a respective envelope for the at least two echo signals may be performed using a Hilbert transform. In some embodiments, the temporal alignment may be responsive to one or more features different from the signal characteristic being interpolated. For example, the temporal alignment may be responsive to estimated envelopes of the existing echo signals. In some embodiments, the generating an interpolated signal by interpolating a signal characteristic of at least two existing echo signals includes interpolating between existing signals from more than one transmit pulse.

In some embodiments, the signal characteristic of at least two of the plurality of echo signals may correspond to at least one of an amplitude, a phase, or both the amplitude and the phase of the at least two echo signals. In some embodiments, the method may further include identifying auxiliary information regarding the transducer array and configuring the interpolating of the signal characteristic based in part on the auxiliary information. In some embodiments, the auxiliary information may include information about the spacing between elements of the transducer array, and the configuring the interpolating may include selecting a number of signals to be interpolated between received echo signals based on the spacing between elements. In some embodiments, the method may further include coherently combining the at least two echo signals and the interpolated signal to generate a beamformed signal. In some embodiments, the generating ultrasound image data may include coupling the beamformed signal to a Doppler processor, a B-mode processor, or both, to generate Doppler image data, a B-mode image data, or both.

The methods described herein may be embodied as circuitry or executable instructions configured to cause an ultrasound imaging system to perform the steps of any of the methods described herein. For example, embodiments of the present disclosure may include non-transitory computer-readable medium comprising executable instructions, which when executed cause a processor of an ultrasound imaging system to perform any of the methods herein.

An ultrasound imaging system according to some examples of the present disclosure may include a transducer array configured to transmit an ultrasound pulse toward a medium and receive ultrasound echoes responsive to the ultrasound pulse and a beamformer configured to receive a plurality of echo signals corresponding to the ultrasound echoes. The beamformer may be further configured to generate an interpolated signal by interpolating a signal characteristic of at least two existing echo signals, wherein the at least two existing echo signals include at least two adjacent echo signals selected from of the plurality of received echo signals and previously interpolated echo signals, and wherein the beamformer is configured to perform the interpolating concurrently with or following temporal alignment of the at least two existing echo signals, the temporal alignment being responsive to one or more features of the at least two existing echo signals. The system may further include a processor configured to generate ultrasound image data based on one or more received echo signals and the interpolated signal.

In some embodiments, the beamformer may be configured to calculate an envelope of each of the at least two existing echo signals and temporally align the at least two existing signals based on the envelopes of the at least two existing echo signals. In some embodiments, the beamformer may be configured to generate the interpolated signal by interpolating a signal characteristic of at least two existing echo signals includes interpolating between existing signals from more than one transmit pulse. In further embodiments, the beamformer may be configured to temporally align the at least two existing echo signals responsive to one or more signal properties different from the signal characteristic being interpolated. In some embodiments, the beamformer may be configured to calculate a displacement vector for a respective envelope of each of the at least two echo signals, weight the displacement vectors according to an interpolation factor, and average the weighted displacement vectors to generate the temporal characteristic of the interpolated signal.

In some embodiments, the system may further include a controller configured to control the beamformer, wherein the beamformer is configured to receive auxiliary information regarding the transducer array from the controller. In some embodiments the auxiliary information may include information about spacing of elements of the transducer array and the beamformer may be configured to interpolate signals in accordance with an interpolation sequence selected based in part on the auxiliary information. In some embodiments, the beamformer may be configured to interpolate a number of signal lines between received echo signals and the number may be selected based on the spacing of elements of the transducer array. In some embodiments, the system may further include an ultrasound probe including the transducer array and the beamformer according to the examples herein may be located in the ultrasound probe.

### BRIEF DESCRIPTION OF THE DRAWINGS

FIG. 1 illustrates ultrasound pulses transmitted from an array of ultrasonic transducer elements.

FIG. 2 is an illustration of beamforming of ultrasound echoes.

FIG. 3 is an illustration of beamforming using elastic interpolation in accordance with principles of the present disclosure.

FIG. 4 is a block diagram illustrating an ultrasound imaging system configured to interpolate echo signals in accordance with the present disclosure.

FIGS. 5A, 5B, 5C and 5D are example graphics illustrating the impact of inadequate sampling on point targets and improvement in results which may be achieved using test system including signal processing of ultrasound pulses in accordance with the present disclosure.

FIG. 6 is a flow diagram of a process for beamforming ultrasound signals in accordance with the present disclosure.

FIG. 7 is another example graphic, illustrating a comparison between results achieved with conventional fully sampled and sparsely sampled signals compared to results achieved with a test system including signal processing of ultrasound pulses in accordance with the present disclosure.

### DETAILED DESCRIPTION

Certain details are set forth below to provide a sufficient understanding of embodiments of the disclosure. However, it will be clear to one skilled in the art that embodiments of the disclosure may be practiced without these particular details. Moreover, the particular embodiments of the present disclosure described herein are provided by way of example and should not be used to limit the scope of the disclosure to these particular embodiments.

Generally described, in delay and sum ultrasonic beamforming systems, ultrasound pulses are transmitted using one or more elements of an ultrasound array and resulting echoes from one or more elements of the array are acquired and digitized by the receive electronics. To form a scan-line representing a single line-of-sight within the image, received echoes from one or more transmit firings are delayed appropriately to compensate for time of flight differences to each location on the line of sight and back again and then summed together with appropriate weights. If the transmit and/or receive apertures are inadequately sampled, then the delay and sum process may cause constructive interference not just at the expected location (the “main lobe”) but also at other steering angles, forming so-called “grating lobes” and introducing artifacts into the resulting image. Such constructive interference is a form of aliasing. To minimize grating lobes, phased array transducers may be manufactured with closely spaced elements at a pitch on the order of ½ wavelength and transmission and reception involves most or all elements within the aperture. In many applications, for instance phased array catheters for intravascular ultrasound (IVUS) imaging, the design of adequately sampled arrays may be technically challenging or costly, and the need to pulse and receive on every element may cause similar issues with system design and frame rates. Therefore, systems and methods for forming an adequate image using more sparsely separated elements and/or fewer transmit/receive may be desirable in the field of ultrasound imaging.

As described herein, interpolation, and more specifically elastic interpolation, may be performed on the received signals (e.g., per channel signal data) to estimate signals for missing elements in the array, such as elements that are not configured to fire in a given transmit or to estimate signals that could have been obtained with a more densely packed array. This approach can be used to increase frame rates and use fewer elements while still generating similar image quality to using all the elements of the array. Additionally or alternatively, by using information about the axial and, optionally, lateral correlation of the echo signals, nominal Nyquist limitations can be overcome and grating lobes commonly associated with under-sampled elements can be suppressed. Such a method may be applied to 1, 1.x and 2-dimensional array transducers and/or beamformers and microbeamformers with under-sampled or sparse apertures. In some embodiments, for example in synthetic aperture systems, elastic interpolation may also, advantageously, be employed to reduce the number of transmit/receive sequences necessary to form an ultrasound image.

Generally speaking, the process of elastic interpolation involves calculating a new signal (e.g., an “interpolated echo signal”) from two or more existing (measured or calculated) signals by 1) time-aligning the existing signals, and then 2) combining the time aligned signals to form the interpolated signal. The time-alignment step may be responsive to features of the existing signals (e.g., a feature of an existing echo signal such as the amplitude, phase, and/or respective envelopes of the existing signal) and may vary from sample to sample along the signal. Additionally, in the context of the present application, the term “existing” signals may be used to refer to signals arising from a single transmit pulse, or they may be signals arising from different transmitted pulses. They may also be interpolated signals (e.g., interpolated echo signals). For example, it may sometimes be desirable to interpolated previously interpolated echo signals and the techniques described herein can be equally applied to such a scenario.

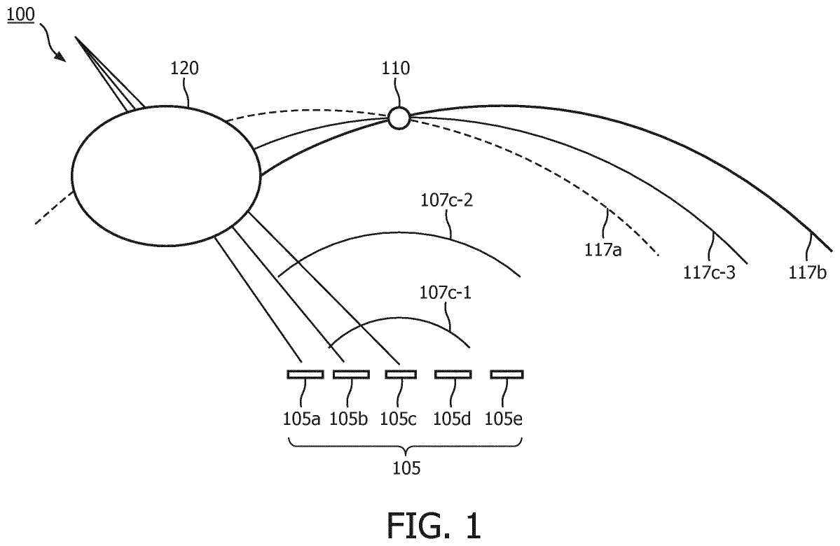

FIG. 1 illustrates wave fronts resulting from sequences of ultrasound pulses transmitted toward a target. In FIG. 1, a target 110 may be imaged by firing elements of a transducer array 105 including individual elements 105a through 105e. FIG. 1 shows the zero phase wave fronts 107c-1, 107c-2, and 107c-3 of an ultrasound pulse transmitted from element 105c of the array towards target 110. When imaging the target 110, elements 105a and 105e may also be fired, e.g., in accordance with a time delay sequence selected to focus the beam at the target 110, and associated zero phase wave fronts 107a and 107e, some of which are omitted to reduce clutter in the illustration, are also shown. On the receive side, received signals from multiple elements are used to focus the receive beam on the target and reduce artifacts in the image (e.g., side lobes or grading lobes resulting from of-axis reflectors, e.g., reflector 120). However, the ability of the signal processing electronics to filter out artifacts is reduced when fewer elements of the array are utilized in any given transmit/receive sequence.

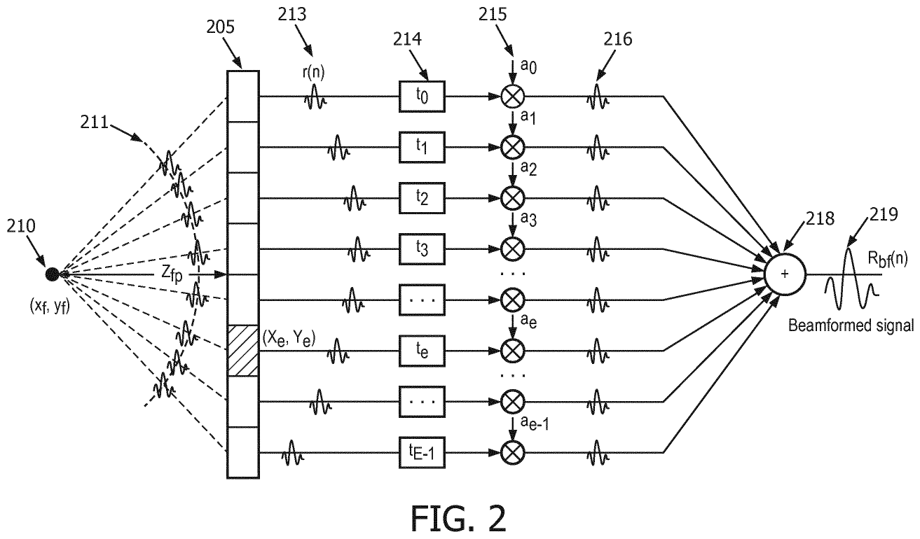

FIG. 2 illustrates aspects of conventional beamforming of signals received by array 205. Echoes 211 resulting from reflector 210 are received by elements of the array 205, and the received echo signals 213 are summed up coherently, that is each received signal is time delayed by a respective delay 214 and weighted by a respective weight 215, and the resulting signals 216 are summed up at 218 to form the beamformed signal 219.

In accordance with principles of the present invention, beamforming (e.g., on transmit or receive) may involve elastic interpolation which involves the adaptive local temporal alignment of signals responsive to the signal properties of the detected signal as part of the interpolation process. In some embodiments, the signal processing steps may include the steps of calculating an envelope for each received echo signal, aligning envelopes of the echo signals for example using an optical-flow based technique, and elastically aligning the signals to the missing line signal. In some examples, calculating the envelopes of each echo may be performed using a Hilbert transform. Alignment of the envelopes may be performed, for example using forward and backward displacement vectors, u(k) and v(k), respectively. The vectors u(k) and v(k) may be calculated to map each sample k from the first signal line to the second signal line and vice versa. Signals from the first and second lines may then be elastically aligned to the missing line location, for example by mapping it to ½ of the forward vector u(k) associated with the first line and ½ of the backward vector v(k) associated with the second line, and then averaging the signals together to form an estimate of the missing line signal.

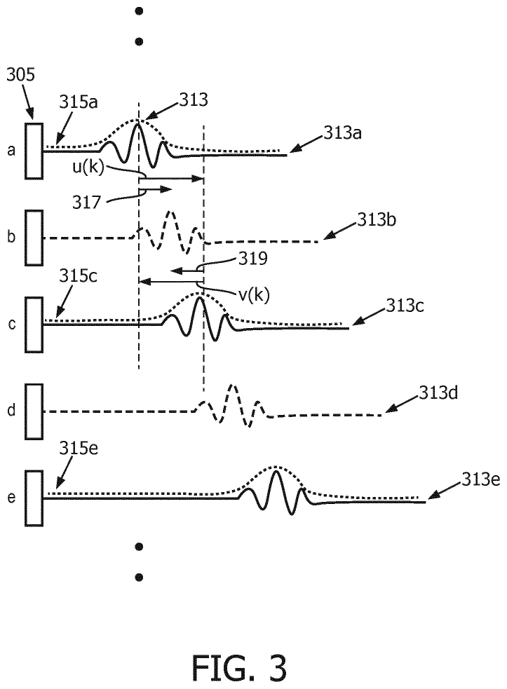

FIG. 3 illustrates aspects of beamforming using elastic interpolation in accordance with principles of the present invention. FIG. 3 shows a portion of an array 305 and the corresponding echo signals 313 received by a subset of the elements (e.g., every other element) of the array 305. The echo signals 313-a, 313-c and 313-e are the pre-time delayed signals (e.g., analogous to signals 213 of the beamformer in FIG. 2) received by the elements 305a, c, and e, respectively of the array 305. In accordance with the present invention, signals from successive echoes may be temporally aligned and then interpolation may be performed to reconstruct a signal from the missing element. The temporal alignment may be responsive to a feature or signal property, e.g., an envelope of the phase or amplitude of the successive echo signals 313-1 and 313-c. For example, feature or signal property such as the signal envelope (e.g., envelopes 315a, 315c, and 315e) may be calculated for each received echo signal using known techniques, for example using a Hilbert transform. Then, forward and backward vectors u(k) and v(k), respectively, may be calculated to map each sample k from a first signal line (e.g., signal 313a) to a second signal line (e.g., signal 313c) and vice versa. As an example, forward and backward vectors u(k) and v(k) may be calculated between the maximum of the envelopes of neighboring received echo signals (e.g., signals 313a and 313c in the illustrated example).

Once the forward and backward vectors between two neighboring received signals have been defined, a missing signal can be interpolated and elastically aligned using the forward and backward vectors u(k) and v(k). Signal characteristics (e.g., signal envelope) for the missing signals may be estimated from the envelopes of the neighboring received signals and temporal characteristics (e.g., the time delay) of the missing signals may be estimated at least in part from the forward and backward vectors u(k) and v(k). The missing signals are thus interchangeably referred to herein as interpolated signals to reflect that they are not received signals but are instead computationally derived. For example, a missing signal (e.g., interpolated signal 313b) may be estimated based, at least in part, on the calculated envelopes of the neighboring received signals 313a and 313c and the forward and backward vectors u(k) and v(k), for example by mapping the maximum of the envelope for the missing signal to a location equal to the average of ½ of the forward vector v(u), labeled as 317 in FIG. 3, plus ½ of the backward vector v(k), labeled as 319 in FIG. 3. Also, while the example herein describes interpolating between the adjacent or successive existing signals (e.g., 313a and 313c), in some examples, the missing signal may be extrapolated from either or both of the existing signals.

Thus, as described, the process of time-alignment may involve, for example with reference to FIG. 3, first calculating the envelopes (315a and 315c respectively) of the echo signals (313a and 313c respectively) and then estimating time shifts (u(k) and v(k) respectively) such that delaying 315a by u(k) results in the peak of 315a aligning with the peak of 315c and delaying 315c by v(k) results in the peak of 315c aligning with the peak of 315a as shown. These time shifts u(k) and v(k) may vary as a function of sample, k, along each echo. In general, the intent is to stretch echo 313a locally to best match echo 313c at all samples and vice versa. In some embodiments, the ranges of u(k) and v(k) may be restricted to maintain the temporal ordering of features along each echo. Other techniques for time alignment may involve, for instance, the adjustment of the phases and amplitude of the respective echoes in lieu of, or in combination with time-shifting.

As will be appreciated, elastic interpolation may be performed in this manner to estimate signals that are missing for any number of reasons, such as due to the use of a sparse array (e.g., where the transducer elements may be spaced farther apart than may be computationally desirable) or due to the use of fewer number of elements for a given firing sequence. It is common to use fewer than all elements in an array in a given firing sequence as it is typical to have a greater number of transducer elements than available signal lines within the receiving electronics (e.g., the beamformer). Thus, the techniques described herein may compensate for loss missing signals regardless of the underlying cause for the missing signals. The signal processing steps described herein may be incorporated within the beamformer, which is typically implemented using one or more application specific integrated circuits (ASICs), field programmable gate arrays (FPGAs) or a high end digital signal processor (DSP) or multiple DSPs. The beamformer processing may be implemented using any combination of hardware and software components as may be suitable for a given application. As will be further appreciated, signals may be elastically interpolated in accordance with the examples herein to include signals for receive beamforming or transmit beamforming. Additionally, interpolation may be performed between signals from more than one transmit pulse. In this manner, interpolated signals for a variety of scenarios may be obtain, including but not limited to interpolate signals from additional (virtual) elements not present in the transducer, interpolate signals from missing, inactive or omitted receive elements, interpolate signals to replace echoes from defective elements, interpolate signals to replace echoes from missing or omitted transmitted pulses.

In some embodiments, auxiliary information may be utilized to enhance the elastic interpolation. For example, auxiliary information regarding the transducer array such as a distance of the spacing between elements of the array may be used to determine how many interpolation steps should be performed to obtain a sufficient quality image and/or determine the factor applied to the vectors, e.g., ½ to both the forward and backward vectors in the illustrated example, but different factors may be used in other embodiments. In some embodiments, multiple missing lines may be interpolated between two neighboring received lines, and a factor other than ½ may be used for the forward and backward vectors at each interpolated line. The factor may depend upon the number of signal lines being interpolated. For example, in a scenario where two missing lines are interpolated, factors of ⅓ and ⅔ (or some other values) of the forward and backward vectors, respectively, may be used for elastically aligning a missing line closest to the first received line and factors of ⅔ and ⅓ (or some other values) of the forward and backward vectors, respectively, may be used for elastically aligning a missing line closest to the second received line. In other examples, the auxiliary information may also include information about the timing and/or sequencing of the transmitted pulses. The elastic interpolation process may be repeated for each pair of neighboring received lines to obtain a signal data set including the original received signals and the interpolated signals. The signals in the signal data set may then be further processed, e.g., temporally adjusted by time delays 214, weighted by weights 215, and summed to obtain an enhanced beamformed signal.

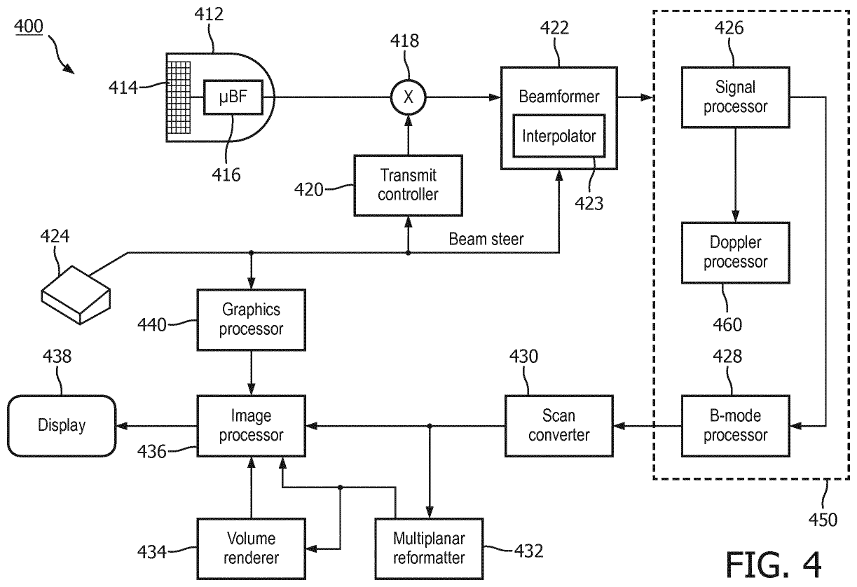

FIG. 4 shows a block diagram of an ultrasound imaging system 400 constructed in accordance with the principles of the present disclosure. The ultrasound imaging system 400 may include a beamformer which is configured to perform elastic interpolation in accordance with the examples herein to produce ultrasound image data. In some embodiments, the beamformer of the ultrasound imaging system 400 may be configured to interpolate a signal characteristic of at least two of the plurality of echo signals to generate an interpolated signal. For example, a signal characteristic of at least two of the plurality of echo signals may correspond to at least one of an amplitude, a phase, or both the amplitude and the phase of the at least two echo signals. A signal characteristic may also be a characteristic derived from the amplitude, the phase, or both the amplitude and the phase, of the at least two echo signals, e.g., an envelope of the amplitude and/or phase of an echo signal.

The ultrasound imaging system 400 in the embodiment in FIG. 4 includes an ultrasound probe 412, which includes a transducer array 414 for transmitting ultrasound waves (e.g., ultrasound pulses which may include focused and unfocused pulses) and receiving echoes responsive to the ultrasound waves. In some embodiments, the array may be incorporated into a transducer probe or it may be an ultrasound patch, e.g., of a flexible array, a large area array, or a multi-patch array. The array of the probe 412 may be configured to transmit any combination of ultrasound pulses, e.g. focused pulses, which may be steered in any desired direction, or unfocused waves (e.g., plane or diverging waves), which may be tilted or angled as may be desired for ultrafast imaging. A variety of transducer arrays may be used, e.g., linear arrays, curved arrays, or phased arrays. The transducer array 414, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction. The transducer array 414 is coupled to a microbeamformer 416, which may be located in the ultrasound probe 412 or other structure (e.g., in the case of an array which is not incorporated into a probe). The microbeamformer 416 controls transmission and reception of signals by the transducer elements in the array 414. In some examples, the array 414 need not be incorporated in a probe but may be the array of a patch, e.g., a single or multi-patch array, which may be configured to at least partially conform to the subject and/or provide one, two or three degrees of freedom of positional adjustability of individual patches.

In some embodiments, the microbeamformer 416 may be coupled by a probe cable to a transmit/receive (T/R) switch 418, which switches between transmission and reception and protects the main beamformer 422 from high energy transmit signals. In some embodiments, for example in portable ultrasound systems, the T/R switch 418 and other elements in the system can be included in the ultrasound probe 412 rather than in a separate ultrasound system base. The ultrasound system base typically includes software and hardware components including circuitry for signal processing and image data generation as well as executable instructions for providing a user interface.

The transmission of ultrasonic pulses from the transducer array 414 may be controlled by the microbeamformer 416, which may be controlled by the transmit controller 420. The transmit controller 420 may be coupled to the T/R switch 418 and the beamformer 422. In some embodiments, the transmit controller 420 may be coupled to the beamformer 422 using a parallel data transfer link which is configured to transmit simultaneously data for multiple or all image lines in a field of view or from multiple or all points within the field of view of the array. The transmit controller 420 may also be coupled to the user interface 424 and receive input from the user's operation of a user controls. The user interface 424 may include one or more input devices such as a control panel, which may include one or more mechanical controls (e.g., buttons, encoders, etc.), touch sensitive controls (e.g., a trackpad, a touchscreen, or the like), and other known input devices.

Another function which may be controlled by the transmit controller 420 is the direction in which beams are steered. Beams may be steered straight ahead from (orthogonal to) the transducer array 414, or at different angles for a wider field of view. In some embodiments, the partially beamformed signals produced by the microbeamformer 416 may be coupled to the beamformer 422 where partially beamformed signals from individual patches of transducer elements may be combined into a fully beamformed signal. Beamforming with elastic interpolation of intermediate signals as described herein may be performed by the microbeamformer, the beamformer, or both. The beamformed signals are coupled to processing circuitry 450, which may include a signal processor 426, a B-mode processor 428, a Doppler processor 460, or combinations thereof.

In embodiments of the present invention, the beamformer 422 (or in some cases, the microbeamformer) may include an interpolator 423 which performs elastic interpolation in accordance with the present examples, e.g., in accordance with the process described with reference to FIG. 3. The interpolator 423 may interpolate a signal characteristic (e.g., an amplitude, phase or a characteristic derived from the amplitude and/or phase, such as an envelope of the signal) and estimate an interpolated signal from (e.g., by elastically aligning the interpolated signal between) at least two echo signals. For example, the interpolator 423 may utilize two neighboring echo signals (i.e., signals received from two adjacent active elements of the array 414) to interpolate a signal that may be received either by an inactive element between the active elements or by a non-existent element that may have been located between the active elements in a denser array. As described herein, the interpolator 423 may calculate respective displacement vectors for each respective envelope of the received echo signals, weight each displacement vector according to an interpolation factor; and average the weighted displacement vectors to align the interpolated signal. In some embodiments, to calcualte the respective envelope for the at least two echo signals, the interpolater 423 may transform each of the at least two echo signals with a Hilbert transform and may then calculate the respective displacement vectors by determining the shift between the maximum of the envelope of the first signal to the second and vice versa. The interpolator 423 may determine a temporal characteristic of the interpolated signal from the displacement vectors. The beamformer 422 may coherently combine the at least two echo signals and the interpolated signal to generate a beamformed signal which may then be coupled to a processor 450, for example the signal processor 426, B-mode processor 428, and/or Doppler processor 460, for generating ultrasound image data. While not depicted in FIG. 4, the interpolator 423 may also be configured to operate within the microbeamformer 416 or another component of the ultrasound imaging system 400.

In some embodiments, the user interface 424 may be configured to display an interface e.g., for receiving instructions for the interpolator 423 or beamformer 422 or microbeamformer 416. The user interface 424 may also be coupled to the beamformer 422 and, thus, coupled to the interpolator 423. The user interface 424 may be configured to provide instructions that control the beamformer, for example to configure the beamformer to receive auxiliary information regarding the transducer array. For example, the auxillary information may include a distance (or spacing) between elements of the transducer array. The user interface 424 may be configured to provide instructions to the interpolator 423 to calculate a time of flight adjustment based on the distance between elements of the transducer array. For example, a user may execute a program at the user interface 424 that provides such instructions to the interpolator 423.

The signal processor 426 can process the received echo signals in various ways, such as bandpass filtering, decimation, I and Q component separation, and harmonic signal separation. The signal processor 426 may also perform additional signal enhancement such as speckle reduction, signal compounding, and noise elimination. The processed signals may be coupled to a B-mode processor 428 for producing B-mode image data. The B-mode processor can employ amplitude detection for the imaging of structures in the body. The signals produced by the B-mode processor 428 may be coupled to a scan converter 430 and a multiplanar reformatter 432. The scan converter 430 is configured to arrange the echo signals in the spatial relationship from which they were received in a desired image format. For instance, the scan converter 430 may arrange the echo signal into a two dimensional (2D) sector-shaped format, or a pyramidal or otherwise shaped three dimensional (3D) format. The multiplanar reformatter 432 can convert echoes which are received from points in a common plane in a volumetric region of the body into an ultrasonic image (e.g., a B-mode image) of that plane, for example as described in U.S. Pat. No. 6,443,896 (Detmer). A volume renderer 434 may generate an image of the 3D dataset as viewed from a given reference point, e.g., as described in U.S. Pat. No. 6,530,885 (Entrekin et al.).

In some embodiments, the signals from the signal processor 426 may also be coupled to a Doppler processor 460, which may be configured to estimate the Doppler shift and generate Doppler image data. The Doppler image data may include color data which is then overlaid with B-mode (i.e. grayscale) image data for display. The Doppler processor 460 may be configured to filter out unwanted signals (i.e., noise or clutter associated with non-moving tissue), for example using a wall filter. The Doppler processor 460 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function and Doppler power estimation is based on the magnitude of the lag-zero autocorrelation function. Motion can also be estimated by known phase-domain (for example, parametric frequency estimators such as MUSIC, ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators.

In some examples, the velocity and power estimates may undergo further threshold detection to further reduce noise, as well as segmentation and post-processing such as filling and smoothing. The velocity and power estimates are then mapped to a desired range of display colors in accordance with a color map. The color data, also referred to as Doppler image data, is then coupled the scan converter 430 where the Doppler image data is converted to the desired image format and overlaid on the B-mode image of the tissue structure containing the blood flow to form a color Doppler overlay image.

Output (e.g., B-mode images, Doppler images) from the scan converter 430, the multiplanar reformatter 432, and/or the volume renderer 434 may be coupled to an image processor 436 for further enhancement, buffering and temporary storage before being displayed on an image display 438. A graphics processor 440 may generate graphic overlays for display with the images. These graphic overlays can contain, e.g., standard identifying information such as patient name, date and time of the image, imaging parameters, and the like. For these purposes the graphics processor may be configured to receive input from the user interface 424, such as a typed patient name or other annotations. In some embodiments, one or more functions of at least one of the graphics processor, image processor, volume renderer, and multiplanar reformatter may be combined into an integrated image processing circuitry (the operations of which may be divided among multiple processor operating in parallel) rather than the specific functions described with reference to each of these components being performed by a discrete processing unit. Furthermore, while processing of the echo signals, e.g., for purposes of generating B-mode images or Doppler images are discussed with reference to a B-mode processor and a Doppler processor, it will be understood that the functions of these processors may be integrated into a single processor.

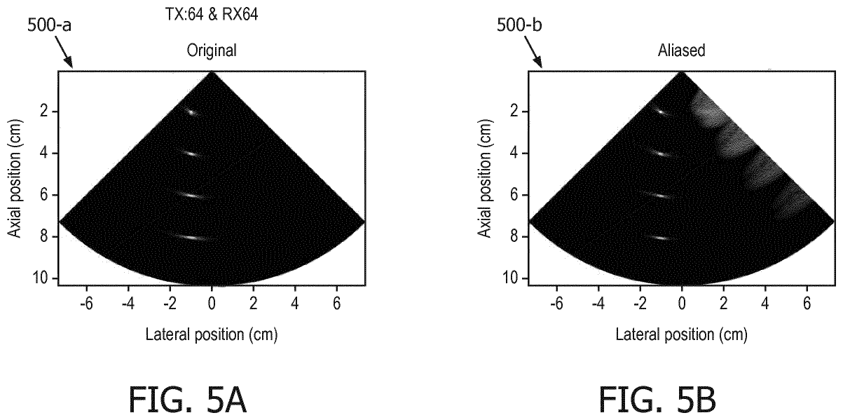

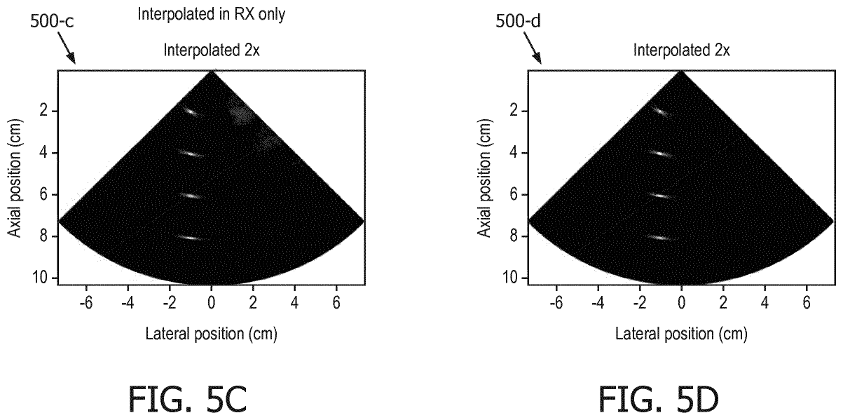

FIGS. 5A-5D show example graphics which illustrate the impact of inadequate sampling on point targets as well as improvements that may be achieved using systems and methods for signal processing of ultrasound pulses in accordance with the present disclosure.

During experimental testing of embodiments of the present disclosure, images showing fully sampled and sparsely sampled conventional arrangements were compared against embodiments in which interpolation was performed on transmit and/or receive in accordance with the present disclosure. FIG. 5A shows a fully sampled example in which the image 500-a was generated using all elements, in this case 64 elements, of the array on transmit and receive. In contrast, the image in FIG. 5B shows an aliased example, in this cases the image 500-b was generated using only half of the elements (for example, every other element) on transmit and receive. As shown, in the image in FIG. 5B, large grating lobe artifacts are visible as areas of increased brightness along the RHS of the image due to the inadequate sample of the echo signals.

FIGS. 5C and 5D show images 500-c and 500-d, respectively, which illustrate example improvements that may be achieved by adding interpolation in accordance with the present disclosure when using an undersampled array. For example, in FIG. 5C, an undersampled array was used (in this case using half or 32 elements of the array) on transmit and receive and interpolation was performed on the received echoes. That is, the 32 received echoes from each from each separate transmit pulse were interpolated to emulate a 64 element receive aperture. As can be observed, the grating lobes are reduced. Some weak grating lobe artifacts may still remain (as visible in FIG. 5C), and further improvements may be achieved by using further interpolation on receive and/or by performing interpolation also on transmit. For example, as shown in FIG. 5D, 32 elements of the array were used on both transmit and receive, but the received echoes have been interpolated to emulate the signals from both the missing receive elements and also the missing transmit elements. In this image, the grating lobe artifacts are substantially fully suppressed providing a significantly improved result over the undersampled examples in FIG. 5B. Although the specific illustrated examples show results from an array having 64 elements and in instances in which half of the elements of the array are used, improvements of this kind can be achieved for virtually any other combination of array with a different number of elements and/or undersampling. Also, it will be understood that while a synthetic aperture using a subset of the elements of the 64 element array was used to demonstrate the principles herein, in practice, the invention could be applied to an array having a sparse array to start with and the interpolation techniques described herein may be used to enhance the image quality that would otherwise be achievable even when using all elements of the sparse array.

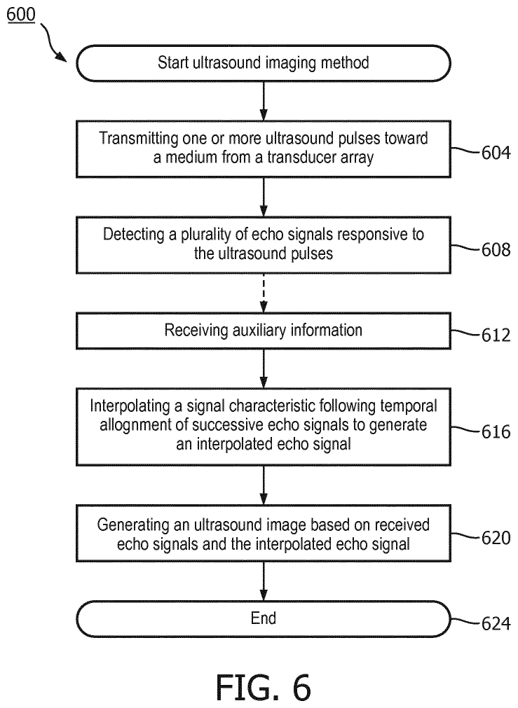

FIG. 6 is a flow diagram of a method 600 for beamforming ultrasound signals including interpolating of signals in accordance with the present disclosure. The method 600 may include transmitting, for example using an ultrasound probe, one or more ultrasound pulses toward a medium (e.g., tissue of a subject to be imaged). The ultrasound pulses may be transmitted in any desired sequence as may be suitable, for example for acquiring B-mode and/or Doppler image data, as shown in block 604.

The method may further include detecting echoes responsive to the transmitted pulses, as shown in block 608. Echoes may be detected using one or more elements of the array. In some embodiments fewer number of elements may be used during a transmit and a receive (also referred to as active elements) than may be physically present in the array. That is some elements may be inactive during any given firing sequence (e.g., transmit/receive cycle). Signals generated by the probe responsive to detected echoes may be referred to herein as received or measured echo signals, while signals generated by the electronics of the probe or ultrasound system (e.g., using interpolation as described herein) may be referred to as calculated echo signals. The received or measured echo signals and the calculated echo signals may collectively be referred to as existing echo signals (that is, existing before a given interpolation step). It will be understood that interpolation according to the present examples may be performed on any two or more existing echo signals, whether measured or calculated.

Referring back to FIG. 6, the method 600 may further include generating an interpolated signal by interpolating a signal characteristic of at least two existing echo signals, as shown in block 616, and generating ultrasound image data (e.g., for generating ultrasound images) based on one or more received signals and one or more interpolated signals, as shown in block 620. As further shown in block 616, interpolation is performed following temporal alignment of successive existing echo signals. As described herein, the interpolating of a signal characteristic may include the calculating (e.g., using a Hilbert transform) of a respective envelope for each of the at least two echo signals and the estimating of an envelope of the interpolated signal by interpolating between the envelopes of the at least two echo signals. The interpolated signal (e.g., the estimated envelope for the missing signal) may then be aligned in temporal relation to the existing echo signals. Signal characteristics that are interpolated according to the present disclosure may be the amplitude of the signal, the phase of the signal, or both the amplitude and the phase of the signals or any characteristics derived therefrom (e.g., an envelope of the signal). When aligning the interpolated signal, displacement vectors are calculated based on the temporal relationship between the received signals and the displacement vectors may be weighted and averaged to derive a temporal characteristic of the interpolated signal, which may then be used to elastically align the interpolated signal.

In some examples, the method may optionally utilize auxiliary information (as shown in block 612), such as information about the distance or spacing of elements in the array. In other embodiments, the auxiliary information may include information about the timing and sequencing of the transmitted pulses. The interpolation may be configurable based on the auxiliary information. For example a different number of signal lines may be interpolated between neighboring received lines if the spacing is greater (e.g., in the case of a sparser array) than when using a more densely packed array and/or when utilizing/activating more elements in a given transmit/receive sequence. Optionally, the interpolation factors (e.g., for weighing of the displacement vectors) may depend upon the auxiliary information. As described, to produce imaging data, the beamforming process may also include coherently combining the signal data set that includes at least two existing echo signals and the interpolated signal to generate a beamformed signal. The beamformed signals may then be coupled to a processor (e.g., a signal processor and subsequently to a B-mode processor and/or Doppler processor) to generate the image data and hence images for display.

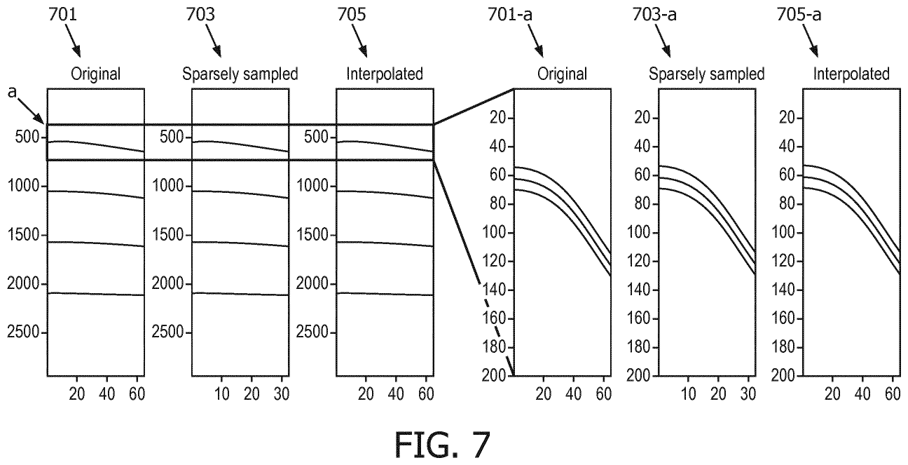

FIG. 7 illustrates further examples of improvements that can be achieved using signal processing of ultrasound pulses in accordance with the present disclosure. In the channel data comparison in FIG. 7, the three columns on the left, and corresponding three columns on the right which show a magnified portion of the left hand side columns, demonstrate the effectiveness of the interpolation techniques described herein, for example in the presence of dominant echoes from isolated targets. The present technique may be well suited for applications where dominant features are present in the received echoes since in such examples alignment may be performed effectively. For example, the techniques described herein may be well suited for applications in intravascular ultrasound, where significant grating lobe artifacts arise from isolated targets such as stent struts and focal calcifications. In such examples, these artifacts may be significantly reduced by the signal processing techniques described herein.

Referring to the example in FIG. 7, a comparison between image qualities for three scenarios is shown. Each of the columns 701, 703 and 705 show the same portion of an image but obtained using a different configuration. Specifically, the image in column 701 was obtained using a reference aperture (e.g., a fully sampled aperture), the image in column 703 was obtained using a sparsely sampled aperture (e.g., the same overall size of aperture but using fewer than all of the elements in the aperture), and column 705 was obtained using the sparsely sampled aperture in the 2^{nd} column but adding signal processing (e.g., interpolation) in accordance with the present disclosure. The right hand side columns, 701-a, 703-a, and 705-a shows the portion a of each corresponding image in the columns 701, 703, and 705 magnified to better illustrate the resolution of the image data obtained in each example. In general, as described herein, an example interpolation technique may be summarized to include the following steps: 1) cross-correlation performed to find the relative time shift between individual channel signals, 2) time-aligning the signals, 3) interpolation performed on the pre-aligned synthetic aperture data in transmit, receive and/or both transmit and receive (e.g., 2× interpolation), and optionally 4) the signals are time-shifted back to the original location. The signal processing technique may include additional or different steps in other embodiments. Also, while generally primarily describing interpolation on the receive beam, similar techniques as described herein may be applied to the transmit beam for transmit beam interpolation to increase the density of a synthetic aperture on the transmit side.

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the disclosure. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A method of ultrasound imaging, the method comprising:
transmitting one or more ultrasound pulses toward a medium from a transducer array;
detecting a plurality of received echo signals responsive to the one or more ultrasound pulses using one or more elements of the transducer array;
generating an interpolated signal by interpolating a signal characteristic of at least two existing echo signals, wherein the at least two existing echo signals are selected from the group consisting of the plurality of received echo signals and previously interpolated echo signals, and wherein the interpolating is performed concurrently with or following temporal alignment of the at least two existing echo signals; and
generating ultrasound image data based on one or more received echo signals and the interpolated signal.

2. The method of claim 1, wherein the interpolating a signal characteristic of at least two existing echo signals comprises:
calculating a respective envelope for each of the at least two echo existing echo signals; and
estimating an envelope of the interpolated signal by interpolating between the envelopes of the at least two existing echo signals.

3. The method of claim 2 wherein the temporal alignment comprises estimating a temporal characteristic of the interpolated signal and aligning the interpolated signal relative to the at least two echo signals based on the temporal characteristic.

4. The method of claim 3, wherein the temporally aligning the interpolated signal comprises:
calculating a displacement vector for a respective envelope of each of the at least two echo signals;
weighting the displacement vectors according to an interpolation factor; and
averaging the weighted displacement vectors to generate the temporal characteristic of the interpolated signal.

5. The method of claim 2, wherein the calculating a respective envelope for the at least two echo signals comprises using a Hilbert transform to calculate the envelope of each of the at least two echo signals.

6. The method of claim 2, wherein the temporal alignment is responsive to one or more features different from the signal characteristic being interpolated.

7. The method of claim 6, wherein the temporal alignment is responsive to estimated envelopes of the existing echo signals.

8. The method of claim 1, wherein the generating an interpolated signal by interpolating a signal characteristic of at least two existing echo signals includes interpolating between existing signals from more than one transmit pulse.

9. The method of claim 1, wherein the signal characteristic of at least two of the plurality of echo signals corresponds to at least one of an amplitude, a phase, or both the amplitude and the phase of the at least two echo signals.

10. The method of claim 1, further comprising identifying auxiliary information regarding the transducer array and configuring the interpolating of the signal characteristic based in part on the auxiliary information.

11. The method of claim 10, wherein the auxiliary information corresponds to a spacing between elements of the transducer array, and wherein the configuring the interpolating comprises selecting a number of signals to be interpolated between received echo signals based on the spacing between elements.

12. The method of claim 1, further comprising:
coherently combining the at least two echo signals and the interpolated signal to generate a beamformed signal.

13. The method of claim 12, wherein the generating ultrasound image data includes coupling the beamformed signal to a Doppler processor, a B-mode processor, or both, to generate Doppler image data, a B-mode image data, or both.

14. A non-transitory computer-readable medium comprising executable instructions, which when executed cause a processor of an ultrasound imaging system to perform the method of claim 1.

15. An ultrasound imaging system comprising:
a transducer array configured to transmit an ultrasound pulse toward a medium and receive ultrasound echoes responsive to the ultrasound pulse;
a beamformer configured to receive a plurality of echo signals corresponding to the ultrasound echoes and generate an interpolated signal by interpolating a signal characteristic of at least two existing echo signals, wherein the at least two existing echo signals are selected from of the plurality of received echo signals and previously interpolated echo signals, and wherein the beamformer is configured to perform the interpolating concurrently with or following temporal alignment of the at least two existing echo signals; and
a processor configured to generate ultrasound image data based on one or more received echo signals and the interpolated signal.

16. The ultrasound imaging system of claim 15, wherein the beamformer is configured to calculate an envelope of each of the at least two existing echo signals and temporally align the at least two existing signals based on the envelopes of the at least two existing echo signals.

17. The ultrasound imaging system of claim 16, wherein the beamformer is configured to generate the interpolated signal by interpolating a signal characteristic of at least two existing echo signals includes interpolating between existing signals from more than one transmit pulse.

18. The ultrasound imaging system of claim 15, wherein the beamformer is configured to temporally align the at least two existing echo signals responsive to one or more signal properties different from the signal characteristic being interpolated.

19. The ultrasound imaging system of claim 18, wherein the beamformer is configured to:
calculate a displacement vector for a respective envelope of each of the at least two echo signals;
weight the displacement vectors according to an interpolation factor; and
average the weighted displacement vectors to generate the temporal characteristic of the interpolated signal.

20. The ultrasound imaging system of claim 15, further comprising:
a controller configured to control the beamformer, wherein the beamformer is configured to receive auxiliary information regarding the transducer array from the controller.

21. The ultrasound imaging system of claim 20, wherein the auxiliary information includes information about spacing of elements of the transducer array and wherein the beamformer is configured to interpolate signals in accordance with an interpolation sequence selected based in part on the auxiliary information.

22. The ultrasound imaging system of claim 21, wherein the beamformer is configured to interpolate a number of signal lines between received echo signals and wherein the number is selected based on the spacing of elements of the transducer array.

23. The ultrasound imaging system of claim 15, further comprising:
an ultrasound probe including the transducer array and a microbeamformer, and
wherein the beamformer corresponds to the microbeamformer.
